(19) United States
(12) Patent Application Publication
(10) Pub. No.: US 2025/0251404 A1
(43) Pub. Date: Aug. 7, 2025

(54) Analytical Platform for Biological Function Appraisal in Recombinant Therapeutic Proteins

(71) Applicant: Novilytic, LLC, West Lafayette, IN (US)

(72) Inventors: Meen Narsimhan, West Lafayette, IN (US); Mary Bower, West Lafayette, IN (US); Nathan Morris, West Lafayette, IN (US); Fred E. Regnier, West Lafayette, IN (US)

(21) Appl. No.: 19/043,500

(22) Filed: Feb. 2, 2025

Related U.S. Application Data
(60) Provisional application No. 63/548,941, filed on Feb. 2, 2024.

(51) Int. Cl. G01N 33/68; B01D 15/02; B01D 15/38; G01N 27/447; G01N 33/58

(52) U.S. Cl. CPC G01N 33/6854; B01D 15/02; B01D 15/3804; G01N 27/44726; G01N 27/44791; G01N 33/582; G01N 2500/20

(57) ABSTRACT

A proteoformic analytical platform and method enables analysis of multiple function-related structural features in single recombinant therapeutic protein (RTP) proteoform, rather than in a family of proteoforms. The platform and method achieve rapid identification and quantification of biological quality attributes (BQAs) of the (RTP), and provides a therapeutic performance appraisal (TPA) that assesses the quality of RTP.

### REFERENCE TO RELATED APPLICATION

This application is a utility filing from and claims priority to U.S. provisional application No. 63/548,941, filed on Feb. 2, 2024, the entire disclosure of which is incorporated herein by reference.

### BACKGROUND

Quality assessment of recombinant therapeutic proteins (RTPs) became part of the FDA biopharmaceutical production approval process circa 1982. This was followed in the 2000 era by a program known as “Pharmaceutical CGMP for the 21^{st} Century—A Risk Based Approach”, in which it was recommended that “Critical Quality Attributes (CQAs)” be genetically engineered into RTPs as a means of achieving “Quality-by-Design (QbD)”. The FDA defines a CQA as “a physical, chemical, biological, or microbiological property or characteristic of a recombinant protein that should be within an appropriate limit, range, or distribution to ensure the desired product quality”. See, Guidance for Industry: Q10 Pharm. Quality System. US Food and Drug Administration: Rockville, MD, (2009), www.fda.gov/downloads/Drugs/Guidances/ucm073517. For the sake of clarity, the above chemical CQAs will be referred to here as chemical structure attributes (CSAs) while biological CQAs will be described as biological quality attributes (BQAs). BQAs differ from CSAs in that the later function collectively in synchrony within an RTP to enhance or diminish the functionality of a BQA functionality.

At present, LC-MS/MS and CE-MS/MS dominate RTP quality management through CSA analytics. The ability to: i) acquire tertiary and quaternary structural data; ii) recognize CSAs operating in synchrony within a proteoform; and iii) quantifying the therapeutic potential of a proteoform family, are major limitations of these MS methods. As an example, U.S. Pat. No. 10,018,635, which issued on Jul. 10, 2018, and is entitled “A Method for Analyzing Samples of Biological Fluid Origin” disclosed one technique for quality assessment in manufacturing but did not address determining the therapeutic potential, be it positive or negative, of proteins in a biological sample. The analytical platform presented in the present disclosure circumvents these problems by appraising the therapeutic potential of mAbs directly through molecular recognition based luminon coding. (The term luminon refers to a fluorescent labeled disease protein mimic). The BQA direct assessment methods outlined in the present disclosure will be a major advance in mAb quality management.

The FDA notes that “controlling manufacturing through timely measurements of the process” is a key component of quality management in the production of mAbs. Moreover, “enhancing understanding and control of the manufacturing process should be consistent with our current quality system: quality cannot be tested into products; it should be built-in or should be by design”, referring again to the FDA quality-by-design (QbD) concept. See, Guidance for Industry: Q10 Pharm. Quality System. US Food and Drug Administration: Rockville, MD, (2009), www.fda.gov/downloads/Drugs/Guidances/ucm073517

As production of mAbs evolved, quality management was found to be more difficult than anticipated. The human genome project revealed that approximately 20,300 genes in humans produce a million or more proteins through variations in gene splicing, alternate start sites, proteolytic processing, and hundreds of in-vivo and in-vitro post-translational modifications. These variables cannot be controlled through QbD structure suggestions alone. The host cell genome and environmental conditions further impact proteoform structure and CSA ratios.

BQA formation is an end-event in mAb synthesis, enabled by coupling individual structural features in a distinct 3D structural pattern within a single proteoform. LC-MS/MS or CE-MS/MS identify CSAs and ratios thereof in recombinant therapeutic proteins (RTP) families fail to detect this higher-level structural organization. To overcome the limitations of these prior techniques, the therapeutic performance appraisal (TPA) assay platforms, methods, and reagents described herein assess the functional quality of RTPs through their ability to either: i) sequester coded mimics of proteins mechanistically involved in-vivo with a disease; or ii) identify possible toxicity or immunogenicity threats.

### SUMMARY OF THE DISCLOSURE

The therapeutic performance appraisal (TPA) assay described herein functions like the idiomatic canary in a mine. It provides an early warning of a problem; the broad objective being to assess in minutes the probability that a recombinant therapeutic protein (RTP) has lost its therapeutic potential or is potentially harmful. That is achieved in-vitro according to the present disclosure by using a luminon mimic of the disease protein target to simulate the in-vivo disease environment. (Luminons are defined herein as fluorescent labeled affinity selectors that recognize and code BQAs in an RTP). Immediate, high affinity recognition of the disease protein mimic by the luminon signifies a high probability of a high TPA.

While proteomics is the large-scale structure analysis of all proteins in a proteome, proteoformics is that of single proteoforms. There is great interest in proteoforms today in that single protein species are frequently involved in diseases. Currently, proteomic and proteoformic analyses are achieved primarily by mass spectral (MS) analyses in the gas phase wherein chemical structure attributes alone are being identified.

The “functional proteoformics” assays described in the present disclosure differ from MS analyses in that: i) a fluorophore tagged luminon mimic of a disease associated protein is used to recognize and bind to BQA surface features of single RTP proteoforms; ii) in a cell-culture type environment; iii) that assesses the therapeutic potential of an RTP through the degree of luminon binding. The luminon coding of BQAs is a simpler, quicker, more definitive way to assess RTP quality than physical identification and quantification of CSA ratios among large numbers of proteoforms by mass spectrometry. Within minutes, a mobile affinity selection chromatography (MASC) platform can assess the probability a coded luminon labeled BQA within an antibody will function therapeutically in-vivo.

Fifty or more mAb proteoforms can be generated in a host-cell expression system, arising from an array of CSA combinations within the proteoform family. A problem in CSA analysis by LC-MS/MS or CE-MS/MS of mAbs is that CSAs can function together to convey biological functionality while others play no role, are toxic, or immunogenic, irrespective of their presence or location in an mAb proteoform family, as shown in the example of FIG. 1. Absent this unique set of CSAs within a BQA in a single proteoform, an mAb will fail to be functional. The therapeutic potential of mAb proteoforms can be assessed directly by analysis of BQAs within proteoforms. For that reason, the BQA assays described herein are based on the analysis of proteoform with a proteoformics quality assessment platform. The function of that platform is to appraise the therapeutic functionality of proteoforms directly in an in-vitro environment that mimics the in-vivo world in which the mAbs are intended to function.

There is a consensus that TPA technology should be easy to use, provide rapid data acquisition, adapt to research and production environments, minimize analysis cycle times, and be easily automated. mAb discovery and production occurs in a time-limited, data-dependent decision-making environment. The luminon coding BQA assays described herein can be automatically executed in-vitro assays at the rate of 100 to 1000 tests per day for weeks in discovery, process monitoring, purification, and formulation.

Embodiments of TPA analytical platforms and methods described herein could function in synchrony with LC-MS/MS or CE-MS/MS systems. Embodiments of the rapid TPA assays described herein can serve as an early warning system that determines when higher order LC-MS/MS or CE-MS/MS are required to understand the source and nature of a problem.

Embodiments of the therapeutic performance appraisal (TPA) platform presented herein have multiple advantages. One is the enablement of rapid mAb quality assessment through molecular recognition of luminon mimics of disease associated effectors and antigens (see FIGS. 2-3). The second is that assays are achieved in-vitro in an analytical platform that mimics the in-vivo cellular assay environment. A third advantage is that the ability to quantify the therapeutic potential of RTPs in minutes will enable a major advance in therapeutic protein analytics. This approach is vastly superior to the slow, costly, physical methods currently used in mAb quality management.

### DESCRIPTION OF THE FIGURES

FIG. 1 is a chart showing chemical, physical and biological quality attributes and their relationships in a monoclonal antibody (mAb) proteoform family. The formation of PTM-based chemical structure attributes leads to an increase or decrease in quality. CQAs can be positive, negative, or neutral. Those followed by a negative sign generally repress biological activity. In contrast, those with a −/+ sign can go either way depending on the specific mAb. The impact of a chemical CQA on BQA activity varies between mAbs. The upper dotted lines indicate a negative impact on quality whereas the lower partially dotted lines indicate the impact of a PTM variant can be positive, negative or neutral.

FIG. 2 is an illustration of performance appraisal methods for determining the performance metric (Pm) of an mAb. The performance metric is defined by the equation Pm=100 (Bal/Cas). Multiple labeling and coding modes are shown that can be used in determining the performance metric (Pm). Through differential fluorophores the ratio of affinity selector tagging agents allows Pm to be determined in either the heterogeneous or homogeneous assay format.

FIG. 3 is an illustration of steps involved in a heterogenous TPA assay of bispecific-mAbs in either the heterogeneous or homogeneous mode.

FIG. 4 is a chart of steps for therapeutic performance appraisal (TPA) assay methods according to the present disclosure.

FIG. 5 is an illustration of TPA platforms according to the present disclosure. Heterogeneous assays are performed in Sector A wherein multiple steps of sample preparation and separation are achieved within a single separation column. Homogeneous assays are performed in Sector B that achieves Ultra-Fast Titer assays in a single sector without any form of analyte separation. Sector C is a non-flow based homogeneous micro-titer well assay platform. Sector D includes sampling systems, pumping systems, power supplies, and fluorescence detectors.

FIG. 6 is an illustration of molecular recognition-based identification of structural regions within an intact protein bearing primary, secondary, tertiary, and quaternary structural features related to a biological function. The constant region in the protein is present in all proteoforms of the protein. Paratrope in the family may have structural features that are both directly and indirectly involved in antibody activity. The illustration shows two types of protein affinity selectors. The constant region affinity selector (Cas) is a synthetic peptide, whereas the bioaffinity selector luminon (Bal) is a synthetic mimic of a natural protein bearing an epitope the mAb was designed to recognize. When a mixture of high-affinity selectors is brought into contact with a mAb they are sequestered in less than a minute.

FIG. 7 is an illustration of a PD-1 (programmed cell death protein 1) activity assay at various molar ratios of antibody to antigen with a chromatogram showing retention times. A PD1 activity assay where formation of the antigen-antibody complex is rapidly detected by a change in the Stokes radius. The top illustration is an illustration of the PD-1 activity assay at various molar ratios of antibody to antigen. The bottom chromatogram shows the retention times of free (Ab), partially PD-1 (H6) antigen-saturated (Ag:Ab) and fully antigen-saturated (Ag2Ab) bound forms of Tislelizumab that were detected using a fluorescent luminon agent specific for the Fc region of the mAb. The inset in the bottom chromatogram shows the linear dynamic range of Tislelizumab. The data show that only the Ag2:Ab form is evident when antigen is in molar excess in the reaction. Negative control: CFF 3 μL plus 17 μL PBSA; antigen: PD1 (H6) 0.5 μg in 20 μL PBSA; antibody: Tislelizumab 0.9 μg in 3 μL CFF plus 17 μL PBSA; binding reaction (molar ratio Ab:Ag=1:2): Tislelizumab in 0.9 μg in 3 μL CFF, PD-1 (H6) 0.5 μg in 5 μL PBSA, plus 12 μL PBSA; binding reaction (molar ratio Ab:Ag=1:0.4); Tislelizumab 0.9 μg in 3 μL CFF, PD-1 (H6) 0.1 μg in 1 μL PBSA, plus 16 μL PBSA.

FIG. 8 are graphs of fluor-dependent, protein-induced signal amplification by a MASC (mobile affinity selection chromatography) luminon by a heterogenous assay. Signal amplification is calculated as mAb area/trough area since trough area quantifies the amount of the molecular recognition agent (Cas) depleted from the mobile phase by binding to the mAb analyte.

FIG. 9 is a graph of fluor-dependent, protein-induced signal amplification for a homogenous assay, showing overlaid chromatograms of three UFT assays of human IgG (hIgG) formulated in CFB, where the molecular recognition agent (Cas) in the mobile phase was conjugated to the indicated fluors. Sample volumes are 10 μL in all cases.

FIG. 10 are graphs of a FcYR1 activity assay where formation of the FcYR1-antibody complex is rapidly detected by a change in the Stokes radius. Panel A is an illustration of the FcYR1 activity assay. In a reaction containing approximately 2-fold molar excess of the antibody, free and FcYR1-bound forms of NIST mAb were detected using a fluorescent affinity selector specific for the Fc region of the mAb. NIST mAb monomer (Ab; ret. time 7 min) and NIST mAb complexed with human FcYR1 extracellular domain (FcR:Ab; ret. time 6.4 min) are clearly distinguishable. The retention time of FcR was determined as 8.1 min by monitoring UV absorbance at 280 nm. Panel B shows a quantification by Proteometer-L MASC luminon assay of the FcR:Ab complex (closed circle), and unreacted (free) Ab (open circle) as a function of NmAb amounts in reaction with a constant amount of FcR. Each injection contained 0.27 μg of FcR and the indicated amounts of mAb. 1.6 μg of mAb is approximately a 2-fold molar excess. Data are the average of triplicate injections.

FIG. 11 are graphs of Cd20 activity MASC luminon assay where the formation of the antigen antibody complex is rapidly detected by a change in the Stokes radius. The top graph is a magnification of the chromatogram between retention time 6 and 13 minutes where free and antigen-bound mab are specifically detected. Cfb components are not detected. The bottom left is the full chromatogram, while the bottom right is a graph showing the increase in fluorescence area at retention time 7.2 min, which corresponds to the amount of ag: ab complex formed, as a function of the amount of rituximab in a reaction containing 0.5 μg vlp-cd20.

FIG. 12 are overlaid chromatograms of UFT repeatability injections with test mAbs. 25 μg of each mAb in CFB was injected (10 μL) within the same sequence. The test mAb sample and statistical values associated with area of the peak are shown for each panel.

FIG. 13 is a graph of Linear Dynamic Range of NmAb and biosimilars in CFB (clarified fermentation broth). The top line is Denosumab (IgG2), the next line is Rituximab (IgG1), the third line is NmAb (IgG1), and the bottom line is Nivolumab (IgG4). The injection volume was kept constant across all the samples to assure that interference from CFB components would be constant.

FIG. 14 is a graph of Proteometer®-CV analysis of NIST mAb (16 μg) in clarified fermentation broth (16 μL).

FIG. 15 is a bar graph of total area response for charge variant analysis of NIST mAb in L-His and clarified fermentation broth.

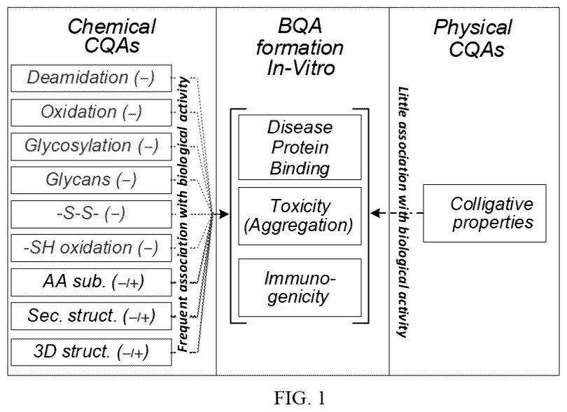

### DEFINITIONS

According to the present disclosure, the following terms are defined as set forth below.
AF647—AlexaFluor647™ is a fluorescent dye that exhibits no increase in fluorescence upon contact with an mAb surface.
Affinity selector luminon—is a mimic of a disease related protein being targeted by a therapeutic monoclonal antibody.
Aggregate—two or more molecules associated in an intermolecular complex.
Analyte—a proteoform family, a family subgroup, or a single proteoform for which a measurement is being made.
Analyzing—refers to an TPA (see definition below) assay wherein two or more features of a coded mAb are being assessed.
Antigen—in the case of TPA assays, it is the disease target to which an antibody binds.
Biologically active mAb—refers herein to a therapeutic antibody that performs its intended function in-vivo.
Biological attribute luminon (Bal)—an affinity selection mimic of an antigen.
Biological performance appraisal—assessing the ability of a mAb to sequester disease associated luminons or mimics thereof.
Biological performance metric—a standard by which the activity of a therapeutic antibody is assessed, which is generally the faction of a mAb that binds a luminon mimic.
Biologically inactive mAb—an antibody lacking the ability to function as a therapeutic agent.
Biological critical quality attribute—properties of a therapeutic protein or mAb that impacts its in-vivo performance.
Bispecific antibodies—a Bs-Ab is a synthetic antibody capable of interacting with two disease related macromolecules.
BQA—a biological quality attribute.
CE—capillary electrophoresis.
CE-MS/MS—capillary electrophoresis-multidimensional mass spectrometry.
Chemical structure attribute—refers to a CSA.
Chemical critical quality attribute—is designated to be a chemical structure attribute referred to as a CSA.
CHO cells—an abbreviation for Chinese-hamster ovary cells.
Constant region affinity selection (Cas)—a luminon that binds to an mAb within a constant structural region.
Continuous process verification or validation—continuous assessment of process continuity.
Critical structure attribute—a structural feature that either positively or negatively impacts monoclonal antibody quality.
Therapeutic performance appraisals—the process of assessing the degree to which therapeutic agent functions as designed.
Critical quality attributes—are molecular features within a therapeutic agent that contribute to its efficacy.
CQA—a critical quality attribute of a recombinant therapeutic protein.
CSA—a critical structure attribute of a recombinant therapeutic protein.
Dimer—a term used herein to specify two antibody molecules joined by adsorption of covalent association in a macromolecular complex.
Disease protein—a macromolecule from a disease cell involved mechanistically in a disease.
ELISA—stands for “enzyme-linked immunosorbent assay”.
Epitope—the structural portion of an antigen to which an antibody attaches.
Fab domain—the region of an antibody that binds to antigens.
FITC—fluorescein isothyocyanate is a fluorescent dye that exhibits an increase in fluorescence upon contact with an mAb surface.
Functional proteoformics—the detection and quantification of BQA bearing proteoforms.
Heterogeneous TPA assays—assays performed in the TPA mode in which there is a separation of luminon coded proteoform products.
Homogenous assays—assays performed in the TPA mode in which there is no separation of luminon coded proteoforms.
Host-cell proteome—the family of non-antibody proteins produced by a cell culture in a fermenter.
LC-MS/MS—liquid chromatography-multidimensional mass spectrometry.
Limit of detection (LOD)—refers to the lowest concentration of an analyte that can be detected.
Limit of quantification (LOQ)—refers to the lowest concentration of an analyte that can be quantified.
mAb—herein refers to a genetically engineered monoclonal antibody.
Metabolome—the family of metabolites produced by a cell culture in a fermenter.
Molecular recognition—a complimentary, non-covalent, structural interaction between molecules
Monomer—is used herein to specify a single antibody molecule.
Microwell array platform—either a microtiter plate or system of small wells in an array that allows simultaneous TPA assays.
Non-separation based TPA assays—these are homogenous TPA in which there is no proteoform separation component.
Paratope—that portion of an antibody that binds an antigen.
PBSA—100 mM sodium phosphate buffer (pH 7.2) containing 150 mM NaCl
Performance metric (Pₘ)—the percentage of all mAb proteoforms in an in-vitro assay that are functionally active.
Proteome—the protein expressed by a cell in-vivo, including those proteoforms that are post-translationally modified in-vitro.
Proteoformics—a field of proteomics dealing with the structure and biological attributes analysis of proteoforms.
Proteoformics platforms—instrument systems that perform proteoform specific assays for BQAa and CSAs.
Proteoforms—protein isoforms of both genetic and post-transformational origin that produce a family of structural variants.
Post-translational modifications—biological modifications that occur in proteins during or after expression.
Protein-initiated fluorescence enhancement (PIFE)—a phenomenon in which a fluorophore bound to a ligand interacts with the surface of a protein after luminon associates with the protein, the net effect being an amplification of fluorescence emission.
Rapid quality appraisal—rapid critical quality attribute assays used in assessing the reproducibility of RTP manufacturing.
Radio-immunoassays—a competitive binding assay between an analyte and radioactive standard.
Recombinant therapeutic protein (RTP)—a protein produced by genetic engineering for the purpose of treating a disease.
Separation-based TPA assay—methods requiring separation of affinity coded monoclonal antibody products.
Steric inhibition or hinderance—refers to inhibition of intermolecular association based on component size.
Therapeutic monoclonal antibodies (mAbs)—genetically engineered antibodies bearing name suffixes, such as -omab, -ximab, -zumab, and -umab; representing murine, chimeric, humanized, and human antibodies, respectively.
TAMRA—carboxytetramethylrhodamine is a fluorescent dye that exhibits an increase in fluorescence upon contact with an mAb surface.
Titer—is the total concentration of all proteoforms in a protein family.
TPA—therapeutic performance appraisal of a recombinant therapeutic protein.

### DETAILED DESCRIPTION

### TPA Modes

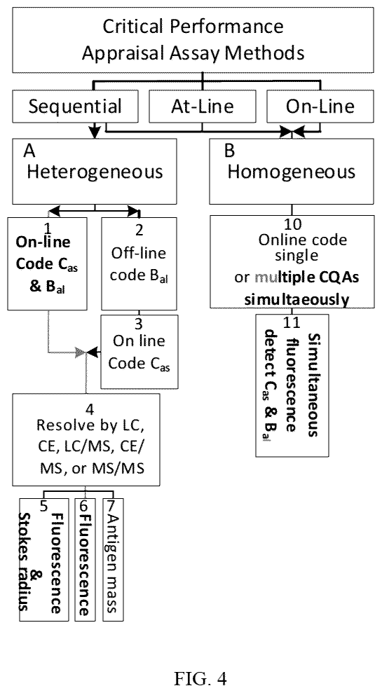

According to the platforms and methods described herein, therapeutic performance appraisal (TPA) assays can be executed in either heterogeneous or homogeneous assay modes, as depicted in FIG. 4. In the heterogenous format of Segment A, the entire assay is carried out in a single mobile affinity selection chromatography (MASC) column that codes and resolves proteoforms based on their Stokes radius. Differentiation between proteoforms is achieved chromatographically based on the Stokes radius of analyte:luminon complexes. A constant CSA structure region common to all the proteoforms in a mAb family is coded with a low molecular weight constant region affinity selection (Cₐₛ) luminon that enables titer quantification in humanized antibodies. Other affinity selector luminons can be used to target biological quality attributes (BQAs) for the purpose of direct appraisal of therapeutic performance. This type of biological affinity selection will be referred to herein as a biological attribute luminon (Bₐₗ).

Analytes are derivatized with the Bₐₗ either off-line (Step 1 of Segment A) or on-line (Step 2 of Segment A) before sample introduction into the liquid chromatography (LC) system of Segment A. Cₐₛ derivatization is always on-line (Step 3 of Segment A). The preferred method of proteoform resolution and detection (Step 4 of Segment A), is by liquid chromatography (LC) or by capillary electrophoresis (CE) with fluorescence (Step 6) or a combination of fluorescence and Stokes radius coding (Step 5).

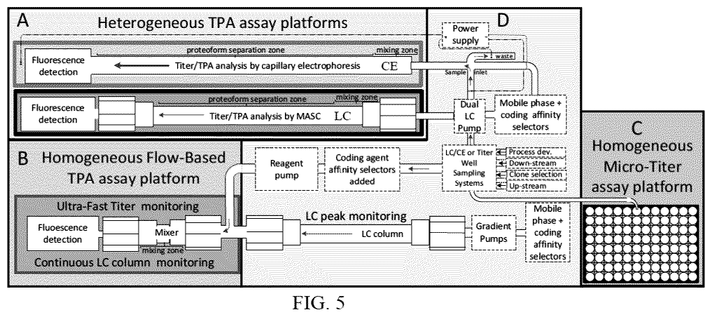

The further mode of TPA is in a homogeneous format, Segment B of FIG. 4. In this mode there is no separation component, as in Step 4 of Segment A. Differentiation between proteoforms is achieved exclusively by differential fluorescent coding of luminons (Step 10 of Segment B) and detection by simultaneous fluorescence (Step 11). All aspects of the assay are performed in an Ultra-Fast Titer (UFT) or a microwell array (MWA) platform (Sector C of FIG. 5). An advantage of this homogeneous assay approach is that titer and BQA assays can be achieved simultaneously without physical resolution of coded mAb:luminon complexes.

### Sample Selection

The TPA technology described herein provides quality assessment of biological samples at all levels of discovery, clone selection, process development, up-stream production, down-stream purification, and formulation.

### TPA Analytical Platforms

Therapeutic performance appraisal (TPA) analytical platforms are disclosed for qualitative and quantitative quality assessment of mAbs in unfractionated biological samples, as depicted in FIG. 5. In all cases, analyte titer quantification is achieved with a constant region affinity selection (Cₐₛ) luminon by fluorescence amplification. The same constant region, fluorophore-specific luminon is used in those cases. Luminon coding and resolution within an LC column or CE column are essential components in heterogeneous assays, Segment A of FIG. 5. Proteoform resolution in the LC platform is achieved by differences in the Stokes radius of proteoforms, while that in the CE platform results from charge differences.

In contrast, homogeneous TPA assay platforms have no separation component, with the Ultra-Fast Titer (UFT) assay platform in Segment B of FIG. 5. In this analytical platform, fluorescent coded luminons in a mobile phase are continuously mixed with samples as they enter the mixing zone of an axial flow mixing component. During passage through the mixer, samples are luminon coded before elution into a fluorescence detector. An essential component of this assay format is that after mixing, fluorescence of the coded luminon is amplified. This amplification of coded luminon fluorescence beyond that of the un-sequestered luminon background enables quantification. The fact that coded luminon is continuously added to the systems and there is no separation component allows the analytical platform Segment B to achieve continuous monitoring. BQA:luminon complex formation occurs during transport through the mixer in Segment B. Constant region Cₐₛ complex formation is achieved in approximately 30 sec. This system can also continuously monitor mAb proteoform elution from a chromatography column.

As an alternative, homogeneous TPA assays can be executed in a titer well formation in Segment C of FIG. 5. In this format, multiple samples are analyzed concurrently. Assays are conducted by addition of reagents, affinity selector luminons, and sample to a microwell in a plate array wherein analyte:luminon complex formation occurs in individual wells. Affinity selector luminons with differentially coded fluorophores are used to target individual CSAs and BQAs by molecular recognition. Titer quantification is accomplished by constant region coding. Within the sample array, internal standard assays are conducted in reference wells for the purpose of residual reagent background subtraction. Quantification is achieved by fluorescence amplification. Quantification of multiple fluorophores is realized by sequential scans of fluorescence emission at different wavelengths. The microwell array is then reconditioned by washing with recycling reagents.

The components in Segment D communicate with the other segments to control the flow of samples, reagents and affinity selectors through the segments. Segment D includes sampling systems, pumping systems and power supplies used by the other segments.

### Analyte Derivatization

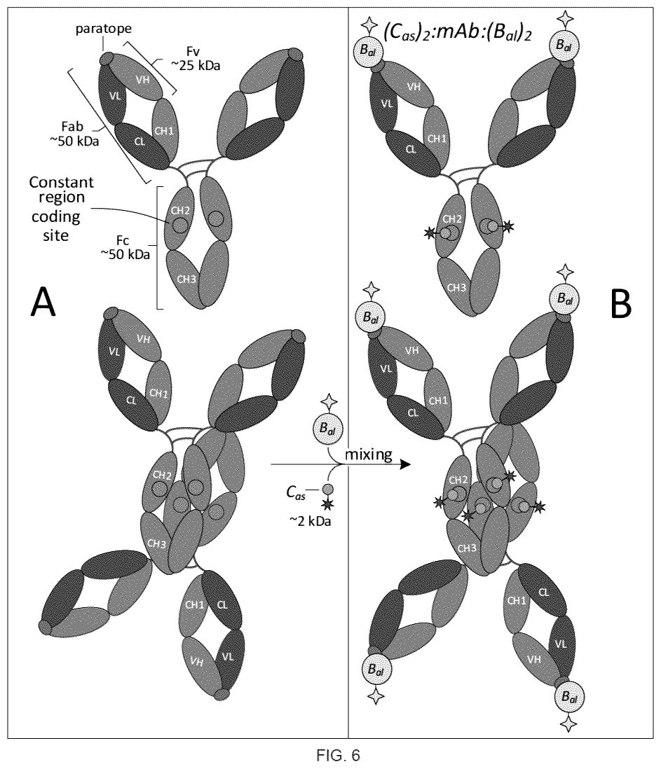

Analyte derivatization with affinity selector luminons is accomplished at multiple sites in proteoforms simultaneously, targeting both constant and variable region CSAs (FIG. 6). That is represented by the reaction

Cₐₛ+Bₐₗ+mAb→(Cₐₛ)₂:mAb:(Bₐₗ)₂+(Cₐₛ)₂:mAb:(Bₐₗ)ₓ
in which the fluorescent labeled luminon, referred to here as constant region affinity selection (Cₐₛ), and a bispecific fluorescent luminon mimic of an antigen or effector function are simultaneously derivatized. There are generally two Cₐₛ whereas the X with Bₐₗ will range from 0 to 2 depending on the degree of mAb activity. The Bₐₗ/Cₐₛ ratio serves as a performance metric as discussed herein.

The potential for mAbs to aggregate and become toxic or immunogenic is also a concern in quality assessment. Differing levels of aggregation arise from varying degrees of surface hydrophobicity, post-translational modifications (PTMs), and the occurrence of —S—S— crosslinking between proteoforms. A complication in TPA assays is that aggregates can differ in CSA content, 3D structure, potential toxicity, and immunogenicity. Since aggregates are often removed in purification, luminon based TPA assays can track aggregation through titer and performance metric assays at all levels of discovery and production (FIG. 6). A dimer, for example, can be a mixture of adsorbed and covalently linked proteoforms that vary in paratope performance. Ideally, a therapeutic antibody would have no aggregates.

Some level of mAb and luminon mixing begins at the analytical platform inlet in both the heterogeneous and homogeneous assay platforms, Segments A and B in FIG. 4. Analytical platforms in the heterogeneous system are packed-bed MASC or conventional LC columns, an open tubular CE capillary column (Segment A in FIG. 5), or some form of permeable polymer matrix. Mixing in mixing zones of pre-separation coding columns is driven by differences in the linear velocity of reagents, analytes, and mAb complexes within the separation platform. There is no separation of excess reagents from (Cₐₛ)₂:mAb:(Bₐₗ)₂ and (Cₐₛ)₂:mAb:(Bₐₗ)₄ complexes before detection. Differentiation between proteoforms is enabled by the properties of the Bₐₗ fluorophores.

Molecular Recognition of Therapeutic Performance Features in mAbs

In 1956, solid-phase extraction, molecular-recognition (Mᵣ) analytics was introduced to clinical diagnostics through solid-phase radio-immunoassays (RIAs). This was followed by the launch of multiple enzyme-linked immunosorbent assay (ELISA) methods. These Mᵣ assays are designed to quantify a single antigen using an antibody to recognize and sequester the antigen by solid-phase extraction. With ELISA assays quantification of the extracted antigen was achieved with a second, enzyme labeled antibody.

Although the therapeutic performance appraisal (TPA) assays described herein also have a molecular recognition component, they are quite different from RIA and ELISA assays. First, RIA and ELISA involve antigen extraction by an insoluble solid phase bearing an immobilized antibody. The TPA assays disclosed herein, in contrast, perform all aspects of an assay in a flowing stream of reagents. Second, antigens (or other disease target proteins) are reagents in TPA assays, not analytes as in immunological assays. Third, multiple critical quality attributes (CSAs) of an analyte family are being examined in TPA assays. Fourth, luminon ratios are used to quantify the ratio of inactive to active mAb proteoforms. And fifth, the biological quality assessment (BQA) assay described herein are a biological performance metric (Pₘ).

### Coding Modes in Molecular Recognition Assays

Fc constant region coding has been proposed to fluorescent code a single Fc constant region structure domain of a genetically engineered therapeutic IgG₁, IgG₂, or IgG₄ mAb for the purpose of quantifying all proteoforms in the family without regard to the rest of their structure. This type of single attribute assay has been referred to as a “Luminon Assay” based on detection by fluorescence. The system and method disclosed herein differs in being the first description of analytical platforms and methods that enable direct Therapeutic Performance Appraisals (TPAs). That assessment is based on comparing biological quality attribute (BQA) analyses with the antibody titer via the equation Pₘ=100 (Bₐₗ/Cₐₛ, where Pₘ is the percent of the antibody that performs as designed, Bₐₗ is the bio-affinity luminon concentration and Cₐₛ is constant region affinity selector concentration.

Pre-coding heterogenous TPA assays according to the present disclosure differentially derivatize a mAb with a constant region affinity selector luminon (Cₐₛ) and BQAs with biological affinity selector luminons (Bₐₗₛ) by molecular recognition and resolution of luminon coded proteoforms by MASC or CE. Proteoform:luminon complexes thus coded are then partially resolved by some form of liquid chromatography (LC) or capillary electrophoresis (CE) method. (See FIGS. 4-5). In a preferred embodiment, coding agent addition to the mobile phase, sample and coding agent mixing, derivatization of BQAs, and partial separation of the coded complexes is accomplished during transport through a single molecular sieving column as in Segment D of FIG. 5. All aspects of this proteoformics assay, except detection, are achieved in a single LC or CE column. Second, elimination of solid phase extract in the BQA analysis format precludes the need for analytical platform recycling between samples. Third, through highly selective fluorescent luminon coding BQA assay can be executed in the presence of non-analytes in the co-eluting proteome, interactome, nucleosome core particles, and metabolome.

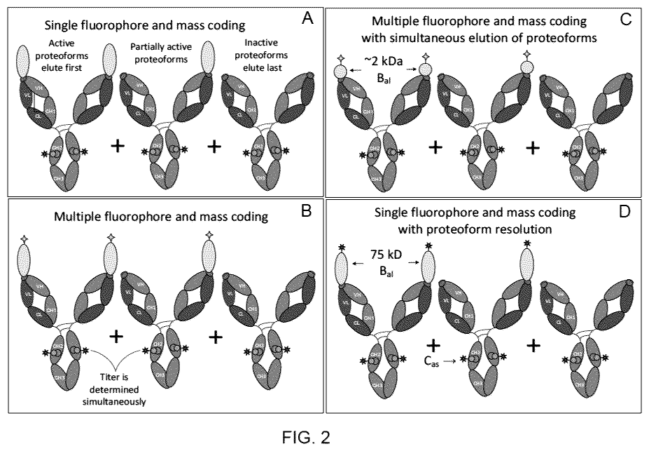

(Cₐₛ)₂:mAb:(Bₐₗ)₂ and (Cₐₛ)₂:mAb:(Bₐₗ)ₓ complex formation in TPA assays occurs in the presence of thousands of other proteins and metabolites of similar fluorescence properties. Based on the broad distribution of tryptophan, tyrosine, and phenylalanine in proteins, direct fluorescence quantification of mAbs in biological samples is precluded. This problem is addressed in three ways, as depicted in FIG. 2. One way is by enhancing detection of the Cₐₛ and Bₐₗ affinity selectors through affinity luminon coding with fluorophores not found in native proteins. As illustrated in Blocks A and C of FIG. 2, a different fluorophore is used with each type of affinity selector luminon. Three to four types of CSAs are quantified simultaneously with affinity coded luminons.

A second mode of coding is to fluorescence code the constant region affinity selection (Cₐₛ) and molecular weight code the Bₐₗ as in Block A of FIG. 2. Given that the molecular weight of an mAb is approximately 150 kDa, binding two disease associated Bₐₗ targets of ˜75 kDa increases the mAb:(Bₐₗ)₂ complex to ˜300 kDa. With one inactive site the molecular weight of the complex is ˜225 kDa. When both sites are biologically inactive the mAb M_{w} remains is ˜ 150 kDa—i.e., the mAb paratope does not bind an antigen mimic. mAb monomer and mAb:(Bₐₗ)ₙ complexes differing in M_{w} are separated by size in a high-resolution MASC molecular sieving column of 30 cm length. Coding differentiation between CSAs is also possible with a single fluorophore when using a high molecular weight fluorescent labeled Bₐₗ, as shown in Block D of FIG. 2. The MASC column is referred to as a sieving column since some luminons and host cell proteins interact weakly with the column surface and may not be strictly separated by size exclusion.

Performance metric (Pₘ) assays in the homogeneous, continuous flow sensor mode (Segment B of FIG. 5) are achieved exclusively with a fluorescent labeled low molecular weight Bₐₗ, as depicted in Block C of FIG. 2. MAb monomer and aggregate content cannot be assessed simultaneously in this assay format.

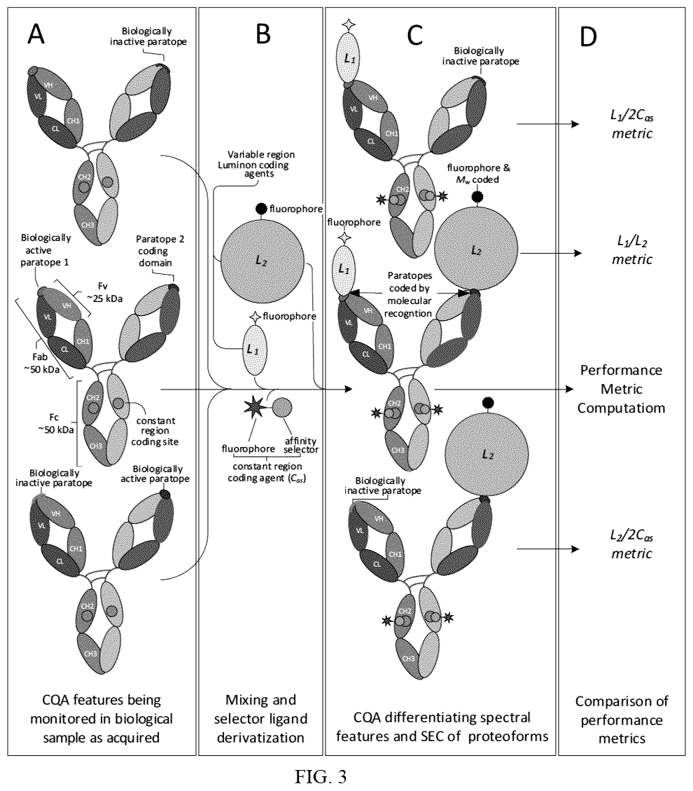

Bispecific monoclonal antibodies (Bs-mAbs) have two paratopes of differing selectivity, although the Fc constant region domain is the same for both halves of the antibody. This allows constant region coding with the same Cₐₛ luminon used in other TPA assays (see FIG. 3). A single variable region affinity selector codes both halves of the Bs-mAb.

Bs-mAb quality is assessed in multiple ways. One approach is by the L₁/L₂ ratio in heterogeneous TPA assays, where L₁ and L₂ represent luminon 1 and luminon 2 respectively (see FIG. 3). The ratio should be constant and near unity in the ideal case, so drift in quality is easily sensed by ratio changes. Failure of either paratope to associate with its target antigen is a warning of changes in potential therapeutic efficacy. The preferred embodiment in TPA assays is to code the constant region CSA and the two paratopes of a Bs-mAb with different fluorophores. Differential paratope coding is required because either of the paratopes can vary. Variants are coded by differences in luminon size, charge, and fluorophores (FIG. 3). This allows L₁/L₂, Cₐₛ/L₁, and Cₐₛ/L₂ metrics to be assessed simultaneously, yielding broad performance validation across multiple domains in the antibody, which, in turn, allows performance drift to be sensed in a single domain of the mAb.

### Separation Methods Used in TPA Assays

### Heterogeneous TPA Assays

Separation based differentiation between proteoforms in the heterogeneous TPA assay platform is illustrated in Segment A of the system in FIG. 5. In this assay format, mAbs are rapidly derivatized at the column inlet by low molecular weight, synthetically coded affinity selector luminons. Proteoform complexes thus formed are resolved during transported through the MASC sieving column. Analyte:luminon complex formation occurs before chromatographic resolution of the coded proteoforms. This means that luminons sequestered in the mAb complex can impact the chromatographic properties mAb:luminon complex. The size of the luminon can also be used to code the site in the mAb being interrogated.

### Post-Separation TPA Assays

A second approach is to execute the chromatographic separation of proteoforms first, followed by luminon coding of mAbs as they elute through a post-column mixer during transport to the detector. This can be designated “Continuous TPA Monitoring” in the homogenous TPA assay format of Segment B of FIG. 5. Beyond reversing the order in which derivatization and separation occurs, proteoform separation occurs before derivatization. That means that luminon coding has no impact on proteoform separation in the continuous column elution format. The fluorescence amplification aspect of the assay is still the same. Charge variant and Stokes radius analyses are preferred embodiments of Continuous TPA Monitoring (see FIG. 9), but other separation modes are possible.

This second detection mode is used in many ways. One is to trigger a fraction collector in process scale chromatography. Effluent from a process scale chromatography column passing through a continuous TPA monitor senses the elution of mAb proteoforms and triggers their collection. In still another mode, effluent from a continuous harvesting fermentor is constantly monitored.

### Detecting In-Vitro Luminon Mimics in TPA Assays

Therapeutic performance appraisal (TPA) assays assess CSA quality indirectly through their association with an in-vitro mimic of an in-vivo disease protein target (see FIGS. 2, 3, 6, 7). Preferred mimics are synthetic luminons designed to recognize and stoichiometrically associate with CSAs spontaneously upon contact. The rationale in this approach is multi-faceted. First, solution based molecular recognition of CSAs in native mAbs by affinity selector luminons occurs orders of magnitude faster than CSA detection methods requiring preliminary sample fractionation of analyte fragment, as in LC-MS/MS. Second, luminon assays have unique detection features that facilitate CSA detection in a single proteoform in the presence of thousands of other molecular species. Third, mAb:luminon complex formation and detection requires no fractionation. Fourth, detection and quantification of CSAs involving non-contiguous structural features is enabled. And finally, multiple CSAs can be examined simultaneously.

Based on detection sensitivity, a preferred mode of coding CSAs for performance appraisal is by derivatization with fluorescence labeled affinity selector luminons. The single caveat in this detection mode is the need to discriminate between the fluorescence of coded mAb:luminon complexes and residual fluorescent labeled luminon derivatizing reagent. This discrimination is achieved by fluorescence amplification upon association of the low molecular weight fluorescent luminon with the much larger mAb.

Mechanistically, fluorescence enhancement and fluorescence amplification are different. Two types of fluorescence enhancement have been described in homogeneous immunological assays. One mode of fluorescence enhancement is the case where the probability of detecting an analyte is enhanced by Förster resonance energy transfer (FRET) spectroscopy. In FRET, energy is transferred from one bound luminon (the donor) to a second luminon (the acceptor) on an analyte surface by means of intermolecular long-range dipole-dipole coupling. Energy transfer efficiency is related to the inverse sixth power of fluorophore separation on the analyte surface. FRET increases detection selectivity by the prerequisite binding of the donor and acceptor fluorophores with 10 to 100 nm distance on an analyte surface. These aspects of FRET enhance the probability of analyte detection.

A second mode of fluorescence enhancement is to impede fluorophore motion. When excited with a beam of vertically polarized light, some degree of that polarization is retained by a fluorophore for a finite time. With 90° detection, detection sensitivity is enhanced by slowing molecular motion before loss of this polarization. Homogenous assays in the TPA format achieve this by artificially increasing the size of a fluorescent luminon through mAb:luminon formation. That occurs when a low molecular weight luminon is sequestered by a protein 100 times larger than the fluorophore bearing luminon. This decreases molecular motion of the luminon and enhances the amount of emitted fluorescence reaching the detector.

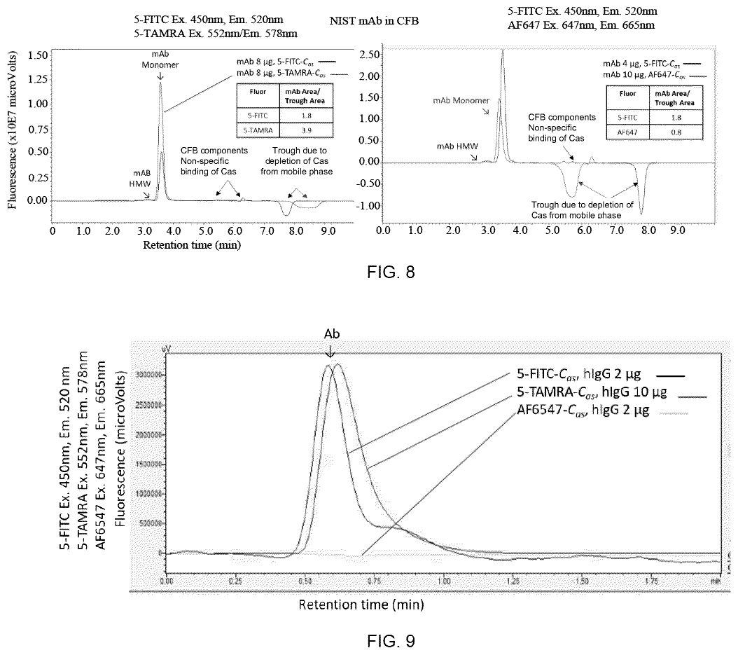

Other modes of enhancement are based on fluorescence amplification upon binding. However, in the current analytical platform, when fluorescent labeled luminons bind to the surface of a protein analyte the fluorophore can bind to the analyte surface as well. This too amplifies fluorophore emission. The degree of amplification depends on the structure of the fluorophore, where it binds on the protein surface, the electrostatic or hydrophobic adsorption mechanism, and solvent displacement at the binding interface. Fluorescence amplification with the constant region luminon is assessed here in the homogeneous assay format as discussed below. When covalently linked to a low molecular weight luminon, TAMRA dye yields the highest degree of fluorescence amplification (see FIGS. 8-9).

### Analytical Platform Embodiments

The present disclosure is not limited to the embodiments described herein, nor to the specific order of steps in which BQA assays are used. Variations in the number and order of steps, addition of other steps, and combinations of the various embodiments herein, fall within the scope of the claims. For example, and by way of illustration, although the embodiments disclosed herein discuss different aspects of the separation, identification, detection, and quantification of mAb proteoforms, these steps can be performed in multiple ways and at various times depending on the proteoformics specific analytical platform, targeting different BQAs and CSAs. Moreover, embodiments of the platforms and methods described herein may be applied to natural or recombinant therapeutic proteins, biomarker peptides, and polynucleotides.

### Heterogeneous Assays

### PD-1 Assay (Antigen Binding Activity Assay)

A function of the mammalian immune system is to clear abnormal cells, including cancer cells from the body. T cells of the immune system infiltrate tumors upon tumor antigen activation, binding the cognate tumor antigen through molecular recognition. This can release cytotoxins from T cells that program cancer cell death. Induction of said cytotoxin release from T cells is mediated through trans-membrane protein expression of programmed cell death protein 1 (PD-1) on the surface of activated T cells.

Cancer cells avert this through a defense mechanism that evades their destruction through expression of programmed cell death ligands 1 (PDL1) and 2 (PDL2). These cancer cell surface ligands bind to PD-1, which blocks T cells cytotoxic activity. MAbs that bind to PD-1 inhibit PD-1/PD-L1 interaction, thereby allowing the body's immune system to fight cancer-almost any cancer. Development of PD-1/PD-L1 targeting mAbs is therefore a focal point of cancer immunotherapies. As of the year 2022, at least 24 types of cancer are being treated with PD-1 targeting recombinant therapeutic proteins.

The proteoformic assay platforms described herein are designed to appraise biological functionality of these RTPs. The objective of the TPA assays devised herein is to assess the probability that a monoclonal antibody(ies) designed to target PD-1 in-vivo will perform as designed, albeit using PD-1 mimics in an in-vitro environment. The TPA assays disclosed herein evolved, in part, from the MASC luminon assay technology described in pending U.S. patent application Ser. No. 18/060,200 (the '020 application), entitled “Molecular Recognition Assays of Critical Structure Attributes in Proteoforms”, filed on Nov. 30, 2022, the entire disclosure of which is incorporated herein by reference. The luminon assays of the '020 application detect and quantify structural features of an antibody. However, one innovation of the TPA assays of the present disclosure is that they assess the biological functionality for which the antibody was designed. Assessing the degree to which an antibody binds to a specific antigen achieves that goal. That appraisal is achieved in-vitro in TPA assays by quantifying the fraction of a therapeutic antibody population capable of sequestering the disease associated antigen, or a mimic.

The ensuing assays are of PD-1 antigen mimics, referred to herein as antibody function or performance assays. It is important to note that in these assays the antibody is an analyte of unknown concentration and the PD-1 mimic is a reagent used at a fixed concentration. Assays are executed in a 7.8 mm×150 mm column packed with 2.7 μm, 300 Å pore diameter hydrophilic media using a mobile phase bearing the fluorescent labeled Cₐₛ constant region luminon in a Proteometer®-L Kit. (The Proteometer®-L Kit sold by Novilytic, LLC, includes reactor, reagent, buffer and reconstitution reagent that transforms any LC into an analytical system capable of identifying and quantifying titer and relative aggregate content in crude culture filtrate in <10 minutes, without sample preparation). Additional components of the mobile phase include 100 mM sodium phosphate buffer (pH 7.2) containing 150 mM NaCl (PBSA) and 5% acetonitrile. Analyses are performed using a standard liquid chromatography (LC) system equipped with an autosampler and a fluorescence detector. The dead volume of the system is less than 40 μL. All runs are of 10 min duration and performed at a flow rate of 1 mL/min and ambient temperature. The autosampler temperature is 4° C. Fluorescence excitation and emission wavelengths are 450 nm and 520 nm, respectively.

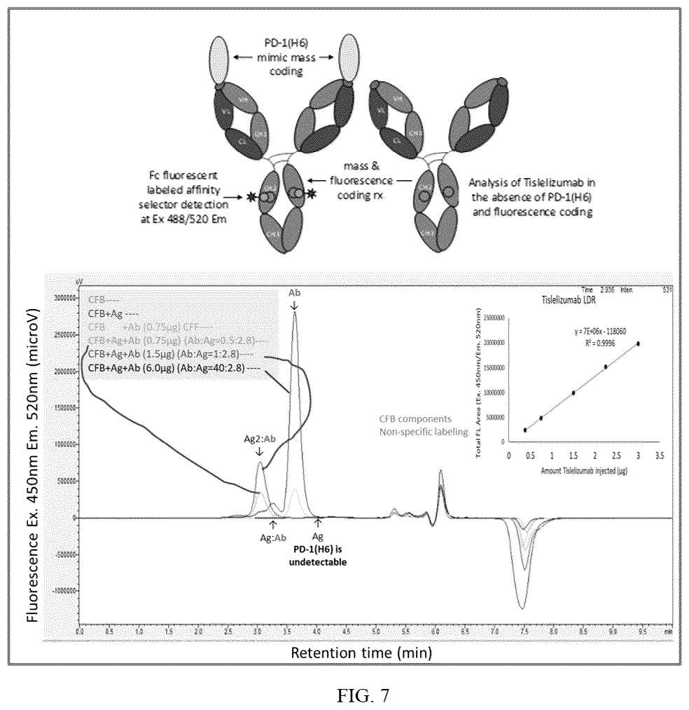

Human PD-1 mimic [PD1 (H6), Leu25-Thr168, with a C-terminal 6-His tag; R&D Systems] is used as an antigen. PD-1 (H6) is formulated in PBSA at a concentration of 0.1 mg/mL for the assay. Clarified fermentation broth (CFB) is prepared from spent growth medium of cultured ExpiCHO-S™ cells (ThermoFisher Scientific) that are grown for 5 days in shake flasks. Tislelizumab, a humanized IgG4 mAb biosimilar for treatment of five cancers that target PD-1 and mimics thereof, is obtained from IchorBio. To mimic fermentor samples, the mAb is formulated in CFB at a concentration of 0.3 mg/mL for an activity assay. Results of a PD-1 (H6) mimic binding assay are shown in FIG. 7. For the binding reactions, the mAb in CFB is mixed with an equal volume of PD-1 (H6) solution in the autosampler and immediately injected. Composition of the injected samples (20 μL) is shown in Table 1 below. The injections corresponding to the chromatogram in FIG. 7 are shown in bolded italics in the table.

TABLE 1
The composition of 20 μL samples used in
TPA assays of a PD-1 Antigen targeting antibody

| | Per injection | | | | |
|---|---|---|---|---|---|
| | Molar | | | | |
| | ratio | Ab | Ag | CFB | PBSA |
| Description | Ab:Ag | μg | μg | μL | uL |
| Blank | | — | — | 10 | 10 |
| Ab | | 0.0375 | — | 10 | 10 |
| Ab | | 0.75 | — | 10 | 10 |
| Ab | | 1.5 | — | 10 | 10 |
| Ab | | 2.25 | — | 10 | 10 |
| Ab | | 3 | — | 10 | 10 |
| Ag | | — | 1 | 10 | 10 |
| Ab + Ag | 0.5:2.8 | 0.75 | 1 | 10 | 10 |
| Ab + Ag | 1:2.8 | 1.5 | 1 | 10 | 10 |
| Ab + Ag | 40:2.8 | 6 | 0.1 | 10 | 10 |

The TPA assay detects mAb: Pd-1 (H6) and free mAb specifically between retention times 2 min and 4.5 min in the MASC column, as shown in the graph in FIG. 7. CFF components and the antigen PD-1 (H6) do not elute in this time window. The Tislelizumab monomer (97.5%, 150 kDa) elutes at 3.63 min and the dimer (2.5%, ˜300 kDa) at 3.13 min (teal line). When the antigen is at a molar excess over the antibody in the binding reaction, as in molar ratio of antibody to antigen 0.5:2.8 and 1:2.8, only fully saturated Ag2:Ab complex is seen at 3.05 min. With a huge molar excess of antibody in the binding reaction, as in molar ratio of antibody to antigen 40:2.8, the partially saturated Ag:Ab complex is seen at 3.27 min and the fully saturated Ag2:Ab complex is seen as a shoulder at 3.05 min. The retention time of the antigen is determined by labeling its HIS tag with HIS Lite™ iFluor® 568 Tris NTA-Ni Complex and monitoring fluorescence at Ex. 555 nm/Em 587 nm.

The inset in graph of FIG. 7 shows that the fluorescent luminon signal for free Tislelizumab in the absence of added antigen has a linear response up to 3 μg. The total area (dimer plus monomer) for 0.75 μg Tislelizumab in the absence of antigen corresponding to peak Ab is 4,830,791 and when this amount of mAb is reacted with a molar excess of antigen, only the Ag2:Ab peak is seen, and its total area is 4,878,196 (Area ratio Ag2Ab:Ab=1.099). For 1.5 μg Tislelizumab plus and minus the same amount of antigen, the Ab and Ag2:Ab peak areas are 9,909,318 and 10,89,341, respectively (Area ration Ag2Ab:Ab=1.099). The data show that specific binding activity of a mAb with its antigen can be determined by the MASC luminon method provided the amount of mAb used is in the linear range of quantitation by the assay and the antigen is present in molar excess to the antibody in the binding reaction.

### FcγR1 Assay (Fc Receptor Binding Activity Assay)

The conserved Fc region in all subclasses of IgGs is responsible for connecting the adaptive immune response with innate immunity. This is achieved by its interaction with extracellular Fc domain receptors present on variety of immune cells, FcγR1 (CD64) being in this group. Its high affinity for monomeric IgG1 (˜10⁻⁸ M) and aggregated IgG1 plays an important role in protection against bacterial infections, binding to IgG1 with 1:1 stoichiometry. FcγR1 also exacerbates certain autoimmune and inflammatory pathologies, particularly in targeting PD-1.

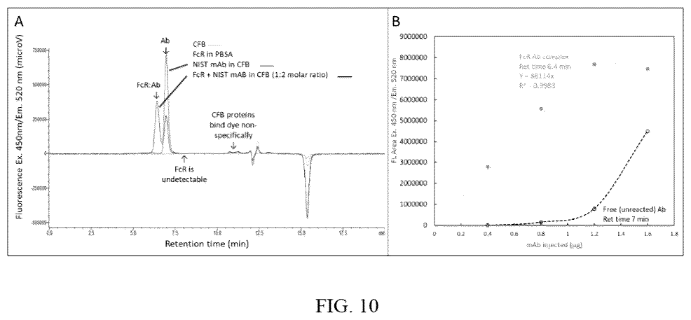

The heterogeneous TPA assay for FcγR1 assay described here is unique in measuring a non-antigen targeting biological activity of an mAb, again in the luminon assay format. The FcγR1 assay is executed as described above for the PD-1 assay, except that the analytes and their concentrations are different, and the flow rate is changed to 1 mL/min. The extracellular domain of FcγR1 (FcR; Gln16-Pro288, with a C-terminal 6-HIS tag purchased from R&D Systems) and NIST mAb RM8671 (Ab) are the analytes. FcR is formulated in PBSA at 0.5 mg/mL. NIST mAb is formulated in CFB at a concentration of 1 mg/mL. Binding reactions contains 0, 3, 6, 9, or 12 μg of NIST mAb mixed with 2 μg of FcR and made up to a final volume of 60 μL with PBSA. The FcR:Ab molar ratio in these binding reactions is 1:0, 1:0.5, 1:1, 1:1.5, and 1:2, respectively. Control reactions lacking FcR are also made. Reaction mixtures are incubated overnight at 4° C. in the LC autosampler for completion of the binding reaction and triplicate injections (8 μL) from each sample are analyzed thereafter. Results of the assay are shown in FIG. 10.

Mab:FcR and free mAb (mAb:luminon) clusters are specifically detected at retention times of 6.4 min and 7 min in the MASC column, as shown in Panel A of FIG. 10. CFB components and the FcR do not elute in this time window. The NIST mAb monomer (97.9%, 150 kDa) elutes at 7 min and the dimer (2.1%, ˜300 kDa) at 6.1 min. When the FcR to mAb molar ratio in the derivatization reaction is 1:2, the free mAb monomer elutes at 7 min, the FcR:Ab complex at 6.4 min, and fully saturates FcR₂:Ab complex at 9.8 min. The small peak at 5.8 min is an FcR-bound aggregate (2.6%). No free Ab is detected at FcR:Ab ratio of 1:0.5 and increasing amounts of free Ab are detected at the higher ratios as shown in Panel B of FIG. 10. The data show that changes in FcR binding specific activity can be detected under these experimental conditions.

### CD20 Assay

CD20 is a B-cell specific marker expressed abundantly by healthy and malignant B mature cells, being a therapeutic target for the treatment of several B cell hematological malignancies. CD20 is expressed at a high level in normal B cells and in virtually all mature B cell lymphoid malignancies. It is absent from pre-B hematopoietic stem cells and terminally differentiated plasma cells. This limits off-target toxicity of mAbs that target CD20 and allows B cell regeneration following therapy.

CD20 is a non-glycosylated cell surface transmembrane phosphoprotein that is predicted to have two extracellular loops and four transmembrane domains, having different epitopes within the extracellular loops that are not necessarily contiguous. Virus-like particles (VLPs) expressing full length transmembrane recombinant human CD20 on their envelopes are optimal, generally having applicable antigens for use in in vitro assays of CD20 binding activity.

The heterogeneous TPA assay method disclosed herein, using a CD20 antigen mimic as the mAb target, is the same as in the PD-1 assay described above. The virus-like particle (VLP) is a large molecule that mimics viruses but is not infectious. A full length human CD20 (70-200 nm) expressed in VLP (purchased from Kactus) is reconstituted at a concentration of 0.2 mg/mL per the manufacturer's instructions. A biosimilar of Rituximab, that is used to treat non-Hodgkins lymphomas is formulated in CFB at a concentration of 1 mg/mL for the CD20 assay. Upon mixing the mAb, CD20 antigen mimic and other requisite reagents, the mAb:CS20 complex is allowed to form spontaneously in 30 minutes at 4 deg C. in an autosampler before analysis.

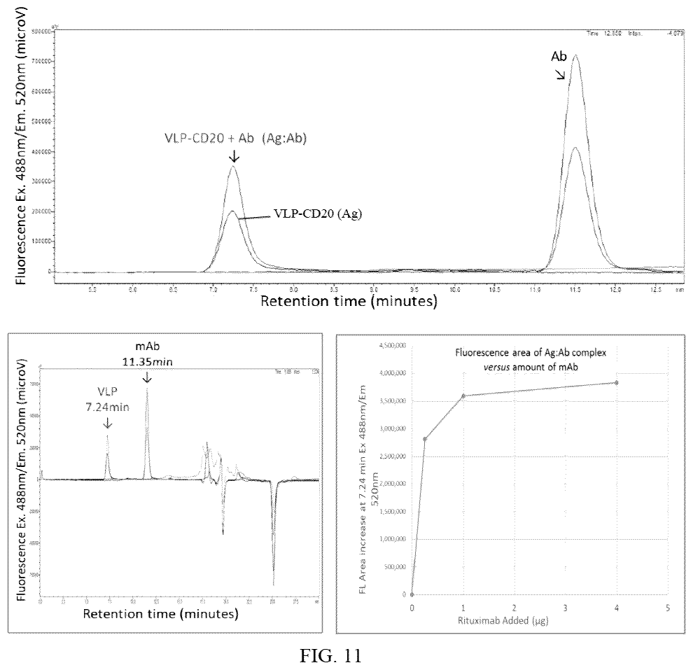

In the heterogenous TPA assay format described herein, constant region fluorescent labeled luminon is constantly added to the mobile phase. In this format, the Fc domain in the mAb: CD20 is detected in the same way, leading to the results shown in FIG. 11. Free and antigen-bound mAb are specifically detected between 6 min and 13 min in the chromatogram. CFB components are not detected in this time window. Rituximab monomer (Ab; 150 kDa, retention time 11.4 min) is seen in CFB bearing samples. VLP-CD20 (Ag, 70-200 nm, >1000 kDa) has a background signal (retention time 7.2 min). When the antigen and antibody are incubated together in the binding reaction, the formation of the Ag:Ab complex is detected as an increase in the peak at 7.2 min with a concomitant decrease in the Ab peak at 11.4 minutes. Increases in fluorescence area at 7.2 minutes correspond to the amount of Ag:Ab complex formed as a function of the amount of Rituximab in a reaction containing 0.5 μg VLP-CD20. Since fluorescence signals can be affected by many factors such as the hydrophobicity of the fluor environment and freedom of fluorophore rotation, the best way to quantify the specific activity of the antibody is to express it as the ratio of the increase on fluorescence at 7.2 min due to formation of the Ag:Ab complex, within the linear dynamic range for Ab quantification.

In this embodiment, the samples examined (20 μL) are:
Negative control: CFB 8 μL in 12 μL PBSA,
Antigen: VLP-CD20: 0.5 μg in 20 μL PBSA,
Antibody 1: Rituximab 4 μg in 4 μL CFB plus 16 μL PBSA,
Antibody 2: Rituximab 1 μg in 1 μL CFB plus 19 μL PBSA,
Antibody 3: Rituximab 0.25 μg in 0.25 μL CFB plus 19.75 μL PBSA,
Binding reaction 1: VLP-CD20 0.5 μg in 2.5 μL and Rituximab 4 μg in 4 μL CFB plus 13.5 μL PBSA,
Binding reaction 2: VLP-CD20 0.5 μg in 2.5 μL and Rituximab 1 μg in 1 μL CFB plus 16.5 μL PBSA, and
Antibody 3: VLP-CD20 0.5 μg in 2.5 μL and Rituximab 0.25 μg in 0.25 μL CFB plus 17.25 μL PBSA.

### Bispecific Antibody Assay

Bispecific antibodies are the new frontier in immunotherapy. Blinatumomab is a bispecific antibody approved by the FDA for the treatment of relapsed or refractory B-cell precursor acute lymphoblastic leukemia. It is a CD19/CD3 bispecific antibody (bsAb) designed in the BiTE (bispecific T-cell engager) format. That is, Blinatumomab consists of one antigen binding domain specific for CD19 that is connected by a linker to an antigen binding domain specific for CD3. It lacks the Fc domain of IgGs. Several bispecific antibodies targeting either CD3 or CD19 are under development.

CD19 is a transmembrane glycoprotein that is expressed almost exclusively in healthy B cells as well as B cell lymphomas and leukemias. It is expressed in low levels in immature B cells, and as such is a prime target for the treatment of hematological malignancies with mAbs. CD3 is a multi-polypeptide cell surface protein that is a component of the T cell receptor that is responsible for activation of their cytotoxic activity. Blinatumomab exerts its anti-cancer activity by binding to CD19 on the cancer cells and by binding to CD3 on T cells, it brings the cancer cells into the proximity of T cells, simultaneously facilitating T cell activation and cancer cell death.

The heterogeneous TPA assay method for CD19/CD3 binding assay is also a modified MASC assay. Except for the reagents used in the binding reaction, the method is performed as described for the PD-1 assay. Site-specific Allophycocyanin (APC)-Labeled, HIS tagged Human CD19 (20-291) protein (MW 47 kDa) is from Arco Biosystems and formulated in PBSA at 0.1 mg/mL. Human CD3E & human CD3D heterodimer protein with a C-terminal human Fc Tag on both CD3E and CD3D (MW ˜100 kDa) is from Arco Biosystems and is formulated in PBSA at 0.1 mg/mL. Blinatumomab biosimilar with a hexa-His tag (MW 54 kDa) is purchased from Ichorbio and formulated in CFB at 0.1 mg/mL. Detection of CD3 is based on fluorescence detection Ex. 488 nm Em. 520 nm of the bound molecular recognition agent. Detection of APC-CD19 is based on APC fluorescence detection Ex. 640 nm Em. 661 nm. Expected results of a bispecific antibody activity assay are described below. For this analysis, the reaction is allowed to proceed for at least 30 minutes in the autosampler at 4° C. before analysis. The injected samples (20 μL) are:
Negative control: CFB 5 μL plus 15 μL PBSA,
Ab: Blinatumomab (Ab) 0.05-0.5 μg in 5 μL CFB plus 15 μL PBSA,
Ag1: APC-CD19 (Ag1) 0.3 μg in 3 μL PBSA plus 17 μL PBSA,
Ag1 plus Ab:Ag1 0.3 μg in 3 μL, Ab 0.5 μg in 5 μL CFB PBSA plus 12 μL PBSA,
Ag2: hFc-tagged CD3 (Ag2) 0.6 μg in 6 μL PBSA plus 14 μL PBSA, and
Ag2 plus Ab:Ag2 0.6 μg in 6 μL PBSA, Ab 0.5 μg in 5 μL CFB PBSA plus 9 μL PBSA.

Complete binding reactions Ab:Ag1:Ag2 (molar ratio Ag:Ab=1:0.17 to 1:1.7): Ab 0.05-0.5 μg in 5 μL CFB, Ag1 0.3 μg in 3 μL PBSA, Ag2 0.6 μg in 6 μL PBSA plus 6 μL PBSA.

Since the assays are performed in the heterogenous TPA assay format, human Fc recognizing fluorescent labeled luminon is constantly added to the mobile phase. This enables detection of free and bound forms of Ag2 at Ex. 450 nm/Em 520 nm, as shown in FIG. 9. Free and bound forms of Ag2 are differentiated by their respective Stokes radii. CFB components do not interfere with the detection of free and bound forms of Ag2. Ab (54 kDa) does not contain Fc and remains invisible. Ag1, on the other hand, is a covalently labeled fluorescent molecule that is detected at Ex. 640 nm/Em. 661 nm. Again, free and bound forms of Ag1 are differentiated by their respective Stokes radii while CFB components and Ab remain invisible.

At Ex. 450 nm/Em 520 nm, free Ag2 (100 kDa) is evident as a peak with retention time around 12.4 min in the Ag2 injection. In the Ag2 plus Ab injection (molar ratio Ag:Ab=1:1.7), only Ab:Ag2 (150 kDa, 11.5 min) is seen. In the complete binding reaction Ab:Ag1:Ag2 containing 0.5 μg (molar ratio Ag:Ab=1:1.7 for each Ag), a peak corresponding to Ab:Ag1:Ag2 (200 kDa, 10.4 min) is the only peak. With lower amounts of Ab in the binding reaction (molar ratio Ag:Ab=1:0.34), both Ab:Ag1:Ag2 complex (10.4 min) and free Ag2 (12.4 min) were seen.

At Ex. 640 nm/Em. 661 nm, free Ag1 (47 kDa) is evident as a peak with retention time around 13.5 min in the Ag1 injection. In the Ag1 plus Ab injection (molar ratio Ag:Ab=1:1.7), only Ab:Ag1 (˜100 kDa, 12.4 min) is seen. In the complete binding reaction Ab:Ag1:Ag2 containing 0.5 μg (molar ratio Ag:Ab=1:1.7 for each Ag), a peak corresponding to Ab:Ag1:Ag2 (200 kDa, 10.4 min) is the only peak. With lower amounts of Ab in the binding reaction (molar ratio Ag:Ab=1:0.34), both Ab:Ag1:Ag2 complex (10.4 min) and free Ag1 (13.5 mmin) are seen.

The ratio of the fluorescence area at Ex. 450 nm to the fluorescence area at Ex. 640 nm for the Ab:Ag1:Ag2 complex peak (10.4 min) is a measure of the biological activity of the bispecific antibody. This ratio is used to compare quality between batches of bispecific antibody, provided the same mass of bispecific antibody in every binding reaction and neither antigen is completely saturated at this mass of antibody.

### Charge Variant Analysis by Continuous TPA Monitoring

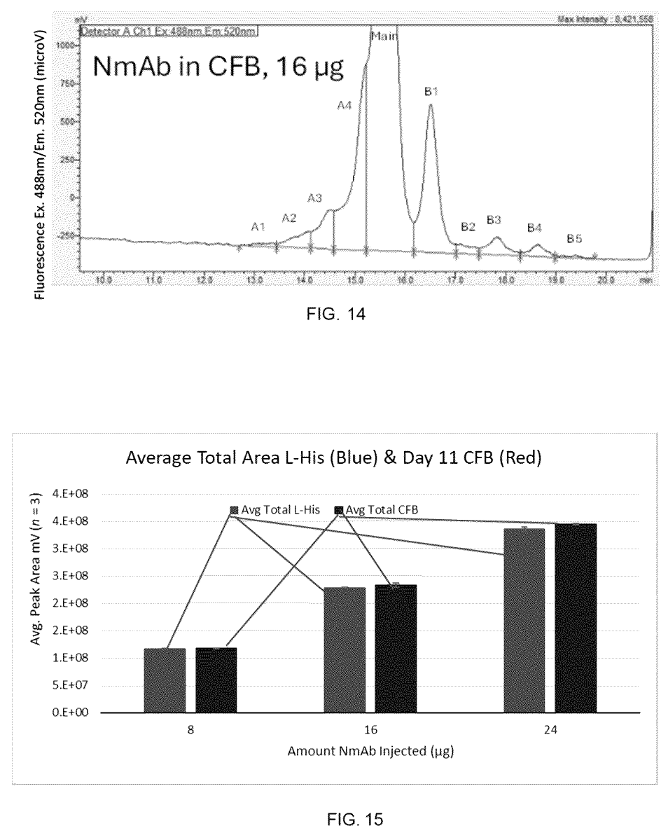

Monitoring throughout the development and production cycle of an mAb is of critical importance as some charge variants arise from post translational modifications (PTMs) of the molecule. Beyond the fact that PTM bearing mAb variants can vary in biological activity, some PTMs convey toxicity or immunogenicity. With mAbs, the most common of these in vitro PTMs are pyroglutamic acid formation at protein N-termini, degradation at N- and C-termini, conformational changes involving sulfhydryl and disulfide bridge scrambling, deamidation, and methionine oxidation; all of which may be detected by charge variant analyses. To demonstrate mAb charge variant analysis by a homogeneous TPA platform, a Proteometer®-CV salt kit (provided by Novilytic, LLC) is employed. The charge variant assay described herein evolved from the ultra-fast titer assay technology described above. NIST mAb formulated in CFB or L-Histidine buffer (12 mM L-Histidine pH 6.0) is fractionated on the Proteometer®-CV column using a 0-14% gradient of mobile Sector B in the platform shown in FIG. 5. Effluent from the Proteometer®-CV column is passed through one arm of a mixing tee, the other arm of which is connected to a secondary pump, and out to the fluorescence detector via a mixer. The secondary pump's mobile phase contains the luminon agent, 5-FITC-Cₐₛ, at a final concentration of 4 μM. An example chromatogram obtained by injecting NIST mAb in CFB is shown in FIG. 14. The data in FIG. 15 show that the fluorescence areas for injections of 8, 16, and 24 μg NIST mAb were comparable, regardless of whether NIST mAb is formulated in buffer or CFB, indicating that CFB components do not interfere in the assay. The homogeneous TPA assay format therefore circumvents the need for purification of the sample by Protein A prior to charge variant analysis. This accelerates the mAb analytics.

### Homogeneous Assays

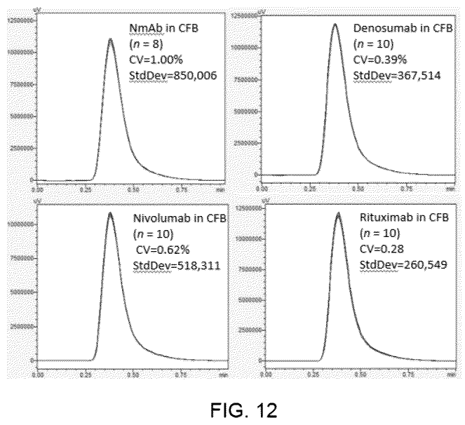

The UFT assay uses a fluorescent detector capable of excitation/emission of 552 nm/578 nm, respectively. Sample introduction is conducted through a primary pumping system in which the mobile phase is PBS (100 mM Sodium Phosphate), pH 7.2/150 mM NaCl)/5% acetonitrile. A secondary pump is used for a mobile phase consisting of 16 μM of 5-TAMRA-Cₐₛ in PBS, pH 7.2/5% acetonitrile. Mobile phases can be prepared daily. A 5-TAMRA-Cₐₛ stock solution is prepared by adding dimethylformamide (DMF) to a determined amount of peptide to generate a final concentration of 1 mM. The peptide is allowed to dissolve for at least 30 minutes at room temperature in the dark before use. To prepare the secondary pump's mobile phase, the appropriate volume of 5-TAMRA-Cₐₛ stock solution is added to PBS/5% acetonitrile solution to obtain a final concentration of 16 μM. One hundred twenty mL of mobile phase allows 140 injections plus at least 20 mL to prime and equilibrate the pump. The mobile phase is pumped through a mixing tee and post-column reactor. Detector sensitivity is optimized to yield the maximum response at the optimal sample loading conditions without saturation of the fluorescent signal. Similar results are seen with four different mAbs (FIG. 12).

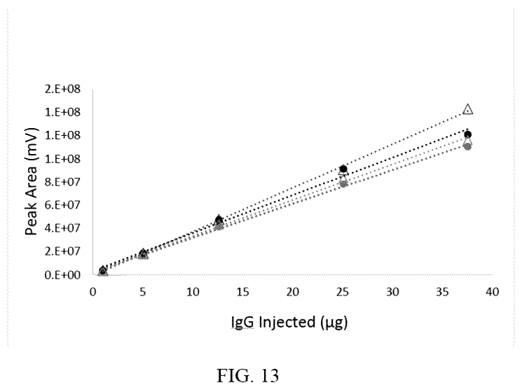

Linear dynamic range studies with biosimilars selected from the three mAb subclasses are executed with samples ranging from 1 μg and 37.5 μg total mAb in 10 μL injection volume (FIG. 13). Method accuracy is assessed by creating QC samples of known concentrations that fall within the LDR of the method (e.g. between 1 μg and 37.5 μg of mAb). The concentration of each QC sample is calculated using the data from the respective mAb standard curve and compared to the theoretical sample concentration. The percent accuracy ranges from 87% with the Denosumab biosimilar to 117% with the Nivolumab biosimilar. Both extreme values are obtained with a sample load 1 μg, near the lower limit of quantification (LLOQ), as reflected in Table 2 below.

TABLE 2
Summary of Experimental Sample Load and Percent Accuracy*

| | 3.5 μg | | 19 μg | | 29 μg | |
|---|---|---|---|---|---|---|
| Theoretical sample load | Estimated | Accuracy | Estimated | Accuracy | Estimated | Accuracy |
| — | (μg) | (%) | (μg) | (%) | (μg) | (%) |
| NmAb in CFB | 3.2 | 91.83 | 19.8 | 105.08 | 29.8 | 102.69 |
| Denosumab in CFB | 4.1 | 17.03 | 20.0 | 106.06 | 33.4 | 115.30 |
| Nivolumab in CFB | 3.1 | 86.98 | 20.0 | 105.47 | 30.0 | 103.95 |
| Rituximab in CFB | 3.3 | 94.58 | 21.1 | 111.59 | 30.8 | 106.09 |

Percent accuracy values were calculated as estimated sample component*100/theoretical sample load.

The UFT assay described herein has utility in a variety of developmental stages of an mAb lifecycle, particularly in an environment where quick, accurate results are of great value. Optimization of culture growth conditions and comparison of productivity of clones are two instances where it can offer considerable time and labor savings. The method involves a very simple experimental setup and requires no specialized instrumentation or training. The linear dynamic range of 1 μg to 37.5 μg sample load is suitable for most sampling conditions and the accuracy offered by the method provides a fast and simple solution to the biopharmaceutical industry in the development and manufacture of therapeutic monoclonal antibodies.

### Fluorescence Amplification

Protein-induced fluorescence signal amplification is central to the homogeneous assays described above. It allows the analyte to be detected against the background of residual fluorescent labeled luminon derivatizing reagent.

The signal amplification phenomenon is explored by a Proteometer®-L type heterogeneous assay, utilizing three different luminon derivatizing agents in the mobile phase. The three luminon agents have the same affinity selection (Cₐₛ) that targets a constant region of human IgGs and differs only in the structure of fluor conjugated to Cₐₛ. The fluorescent labeled luminon is added directly to the mobile phase. The fluor is found to affect binding of the luminon agent to the mAb analyte, and the concentration of the three fluorescent luminon agents in the mobile phase are adjusted accordingly. The mobile phase for 5-FITC-Cₐₛ and AF647-Cₐₛ contains 0.4 UM Cₐₛ, whereas the mobile phase for 5-TAMRA-Cₐₛ contains 2 μM Cₐₛ. Additional components of the mobile phase are 100 mM sodium phosphate buffer (pH 7.2) containing 150 mM NaCl (PBSA) and 5% acetonitrile. Analyses are performed on a 7.8 mm×150 mm molecular sieving column using a Shimadzu LC-40 liquid chromatography system equipped with a SIL-40 autosampler and a Shimadzu RF-20Axs fluorescence detector. The dead volume on the system is less than 40 μL. Data acquisition and instrument control is performed using LabSolutions software. Peak integration is achieved with the i-PeakFinder algorithm. All runs are of 10 min duration and are performed at a flow rate of 1 mL/min and ambient temperature. The autosampler temperature is 4° C.

Signal amplification is examined for NIST mAb formulated in CFB as reflected in FIG. 8. The mAb monomer-Cₐₛ complex and the mAb dimer-Cₐₛ complex peaks (retention times at ˜3.7 min and ˜3.15 min, respectively), as well as the trough corresponding to the amount of luminon agent depleted from the mobile phase due to complexation with the mAb, are seen with all three luminon agents. CFB components have no background. The ratio of the total mAb area (monomer plus dimer) to the trough area is therefore a measure of signal amplification. Signal amplification is greatest for 5-TAMRA-Cₐₛ (3.9-fold), intermediate for 5-FITC-Cₐₛ (1.8-fold) and non-existent for AF647-Cₐₛ.

To demonstrate the importance of signal amplification for homogeneous assays, an ultra-fast titer assay is performed with human IgG (hIgG) formulated at 1 mg/mL in CFB. The UFT assay protocol as described above is performed on the Shimadzu LC-40 liquid chromatography system. The hIgG sample formulated in CFB (10 μL) is introduced via the primary pumping system in mobile phase PBSA (100 mM Sodium Phosphate, pH 7.2/150 mM NaCl) containing 5% acetonitrile. The luminon agent is dissolved in the same mobile phase and introduced by the secondary pump. The concentration of 5-FITC-Cₐₛ and AF647-Cₐₛ in the secondary pump's mobile phase is 8 UM, whereas the concentration of 5-TAMRA-Cₐₛ used is 16 UM. Both flow streams are introduced into the mixer through a mixing tee at a rate of 0.3 mL/min and transported directly into a Shimadzu RF-20Axs fluorescence detector.

In the homogeneous ultra-fast titer assay format (Segment B of FIG. 5), the fluorescent luminon reagent is continuously flowing through the detector at a constant concentration, producing a constant background signal, irrespective of whether it is bound to an analyte or free. To detect an analyte:luminon complex requires an increase in fluorescence after fluorophore binding. An overlay of the chromatographic profile of hIgG in the ultra-fast titer assay with either 5-FITC-Cₐₛ, 5-TAMRA-Cₐₛ or AF647-Cₐₛ luminon agents is shown in FIG. 9. A clear increase in signal is observed with both 5-FITC-Cₐₛ and 5-TAMRA-Cₐₛ. These luminon agents exhibit signal amplification of 1.8 and 3.9 fold, respectively, in the Proteometer®-L type heterogeneous assay platform (see FIG. 8). As expected, no IgG signal is observed in the homogeneous assay with the AF647-Cₐₛ luminon agent (FIG. 9). AF647-Cₐₛ shows no signal amplification (FIG. 8).

What is claimed is:

1. A method for conducting a biological function specific (BFS) proteoformics assay that rapidly identifies and quantifies biological quality attributes (BQAs) in a recombinant therapeutic protein (RTP) proteoform via luminon coding of structural components thereof, comprising:
a) luminon coding of BQAs in the RTP proteoform, by
(i) continuous or sequential addition of an RTP titer into a mobile phase bearing luminon coding reagents that target BQAs, and
(ii) forming BQA:luminon complexes by mixing the RTP sample and the coding reagents in an axial-flow mixer or a mobile affinity selection chromatography (MASC) component, such that the BQAs are luminon coded, and
(iii) transporting the ensuing luminon coded BQA complexes to a detection sector, wherein the RTP titer alone, or in combination with BQAs, are identified and quantified for the purpose of appraising the therapeutic performance potential of the RTP through BQA to RTP titer concentration ratios,
b) detecting, in the detection sector, luminons in the BQA:luminon complexes by either their fluorescence or through fluorescence resonance energy transfer,
c) generating structure specific luminon coding, identification, and quantification of
(i) a constant region BQA in all proteoforms of an RTP family, independent of BQA content,
(ii) secondary BQAs required for an RTP to have specific biological functionality, and
(iii) multiple functional BQAs of either positive or negative BQA functionally,
to thereby appraise the safety and therapeutic potential of the RTP.

2. The method of claim 1, wherein:
prior to the step of transporting the luminon coded BQA complex to a detection means, transporting the luminon coded BQA complex to a separation sector configured to resolve BQA:luminon complexes by either mobile affinity selection chromatography (MASC) or capillary electrophoresis (CE) to identify unique BQA structural features in RTP proteoforms based on their elution position and luminon coding; and
the step of generating structure specific luminon coding, identification, and quantification includes;
a) quantification of BQA:luminon complexes thus resolved by fluorescence amplification of the fluorophore labeled luminons in the complexes, followed sequentially by
b) summing the luminon concentration in all proteoforms eluting from the separation column to quantify the RTP titer, and
c) comparing RTP titer concentration with that of function targeted BQA concentration, to assess the therapeutic potential of a proteoform population as needed in host-cell clone selection, process development, production, RTP purification and/or therapeutic product formulation.

3. The method of claim 1, wherein:
prior to the step of forming BQA-luminon complexes,
a) transporting the RTP sample and the coding reagents through a chromatographic or electrophoretic separation sector, such that luminon coding of BQAs at or before the separation sector will alter and preclude their identification with multiple types of post-translational modifications, wherein analytes elute sequentially from the separation sector, and
b) eluting analytes sequentially from the separation sector to undergo analyte:luminon complexes formation; and
the detecting step includes fluorophore amplification of the analyte:luminon complexes in the detection sector, that undergo; and
the step of generating structure specific luminon coding, identification, and quantification is applied to the analyte:luminon complexes.

4. A therapeutic performance appraisal (TPA) method including reagents that jointly assess the quality of recombinant therapeutic proteins (RTPs) through their ability to sequester coded mimics of proteins mechanistically involved in-vivo with a disease, the method comprising,
a) performing multiple aspects of the TPA assay within a single assay analytical platform, selected from
(i) a mobile affinity selection chromatography column,
(ii) a capillary electrophoresis column, and
(iii) a flow-through mixer,
b) defining the analytical platform as
(i) a heterogeneous TPA assay platform including a chromatographic or electrophoretic separation component that mixes analytes and luminon derivatizing agents at a column inlet to the separation component, causing non-covalent luminon coding of critical quality attributes in analyte proteoforms by molecular recognition, resolving analyte complexes thus coded based on their physical and biological properties, and detecting the luminon coded complexes through their fluorescence properties, or
(ii) a homogeneous TPA assay platform in which one or more critical quality attributes are differentially coded with fluorescent label luminons in a mixing sector wherein RTP:luminon complexes are formed and subsequentially quantified without resolution by fluorescence enhanced detection,
c) choosing a luminon coding strategy that fits the TPA assay platform, selected from
(i) fluorescence coding alone or in combination with either Stokes radius or mass coding in heterogeneous assays,
(ii) fluorescence coding exclusively in homogeneous assays, and
(iii) a combination of fluorescence and mass coding,
d) adding one or more coding luminons to the mobile phase of the assay platform continuously at a point before sample addition for the purpose of rapid, sequential TPA assays of samples through RTP:luminon complex formation,
e) adjusting the mobile phase flow-rate to allow luminon coding of RTPs before elution from the TPA platform sector,
f) selecting multiple, variable biological quality attribute (BQA) features for analysis by luminon coding, selected from
(i) antibody (Ab) aggregation,
(ii) paratopes in mono-specific and bi-specific antibodies,
(iii) Fc effector domains, and
(iv) type specific antibodies,
g) selecting multiple levels of synthetic bioaffinity selector luminon coding that will be used to differentiate between,
(i) Ab analytes and the combined host cell proteome, metabolome, and genome,
(ii) BQA and chemical structure attributes (CSA) targets of bioaffinity selector luminons,
(iii) paratope specific BQAs involved in bispecific antibody quality assessment, and
(iv) constant and variable CSAs for the purpose of determining TPA performance metrics,
h) matching the coding agents to specific synthetic bioaffinity selector luminon mimics of disease associated proteins, selected from
(i) one or more epitope mimics targeting by mono-specific antibodies, and
(ii) double epitope mimics for a single bi-specific antibody,
i) basing the mimic derivatization of an Ab on non-covalent molecular recognition of all types of CSAs,
j) selecting luminon mimics that expedite selective analyte:luminon complex formation in proteome and metabolome rich biological sample based on their analyte specificity and binding constant,
k) coding an accompanying constant region binding site in all proteoforms of the Ab analyte family with a synthetic luminon bearing a unique detection feature that enables titer quantification, by way of
(i) unique fluorescence properties within a low molecular weight luminon, and/or
(ii) separation properties in an LC or CE column,
l) simultaneously quantifying one of more biological quality attributes (BQAs) associated with Ab performance, selected from
(i) attributes associated with multiple paratopes in bispecific antibodies,
(ii) effector domains, or
(iii) aggregation,
m) computing BQA to titer ratios to appraise therapeutic performance metrics for all BQAs being examined,
n) quantifying the performance of both paratopes in bispecific mAbs using differentially labeled epitope mimic luminons that facilitate quality assessment,
o) detecting fluorescence coded antibodies by fluorescence amplification, requiring
(i) selection of a fluorophore showing maximum fluorescence amplification for the antibody domain being targeted, or
(ii) selection of a fluorophore that shows minimal binding to non-analyte proteins, and
(iii) showing maximum sensitivity for the structure region in the Ab being targeted by the fluorescent labeled luminon,
p) quantifying the constant region luminon (CRL) titer through luminon specific fluorophore coding,
q) quantifying each BQA luminon titer in the sample,
r) computing the BQAL to CRT ratio for each BQA luminon to provide a therapeutic performance metric (CPM),
s) computing inter-luminon ratios for all the BQA luminons assayed,
t) time-stamping all data at the completion of each TPA assay,
u) assessing mAb quality and process continuity based on the computations.

5. A therapeutic performance appraisal (TPA) method including reagents that jointly assess the quality of recombinant therapeutic proteins (RTPs) through their ability to sequester coded mimics of proteins mechanistically involved in-vivo with a disease, the method comprising,
a) performing the TPA method within a single assay analytical platform including a microwell array or micro-vial array assay analytical platform,
b) selecting reagents that allow,
(i) preparation of multiple samples for assay through addition of appropriate buffers, internal standards, affinity selector luminons, and sample or internal standards in a specific order to a series of assay wells,
(ii) incubation of samples until the analyte and affinity selector luminons mix with formation of analyte:luminon complexes in-situ,
(iii) preparation of analyte and analyte free samples for a microwell TPA assay,
(iv) quantifying analyte:luminon complexes without resolution, and
(v) achieving the quantification through fluorescence enhancement,
c) recycling the microwell array platform between assays by washing the wells with recycling reagents alone,
d) executing multiple TPA assays together through molecular recognition based coding of analyte features being targeted for identification and quantification,
e) choosing affinity selector luminons with differentially coded fluorophores that match chemical structure attributes (CSA) and biological quality attributes (BQA) binding targets of the RTP, wherein
(i) a fluorescent labeled affinity selector luminon mimics a targeted structural feature or features in the antibody analyte,
(ii) coding is achieved by fluorescence labeling of luminons for the purpose of identifying and quantifying structural and/or biological features of the analyte, and
(iii) affinity selector luminons are coded to match a specific CSA or BQA,
f) labeling a constant region structure feature present in analyte protein a synthetic affinity selector luminon for the purpose of titer quantification,
g) matching the coding agents to specific synthetic bioaffinity selection mimics of disease associated proteins for the purpose of secondary coding, that being
(i) a single protein mimic with a 6× histidine tag that targets a mono-specific feature of an RTP example, or
(ii) a similarly labeled double epitope mimics for bi-specific antibodies,
h achieving analyte quantification by fluorescence enhancement of analyte sequestered luminons, wherein
(i) the quantification of multiple fluorophores is achieved by sequential scans of fluorescence emission at different wavelength, or
(ii) multiple emission wavelengths are monitored simultaneously, and
(iii) background fluorescence from the samples is removed.

6. A method for rapid homogeneous proteoformics assay for rapid identification and quantification of biological quality attributes (BQAs) in recombinant therapeutic protein (RTP) proteoforms via luminon coding of biological function specific structure attributes, comprising:
a) providing an analytical platform having operating sectors capable of luminon coding chemical structure attributes (CSAs) in RTP proteoforms, the analytical platform being comprised of components that sequentially
(i) introduce RTP samples into the platform continuously or as aliquots through introduction into an analytical platform mobile phase bearing BQA luminon coding reagents,
(ii) mix the components in a mobile affinity selection chromatography (MASC) component or an axial-flow mixer, such that luminon coding of RTP BQAs is executed in the flowing mobile phase, wherein the mixer is based on an axial flow device for a homogeneous assay system or differential linear velocity for a heterogeneous assay system, and
(iii) transport the ensuing luminon coded BQA complexes to a detection means in which RTP titer and secondary BQAs that convey biological functionality are identified and quantified, and
b) executing a multi-step BQA coding process within the mobile phase based on
(i) selecting buffers, solvents, and fluorescent labeled luminon coding reagents that enable anayte:luminon coding,
(ii) the luminon coding process uses luminons that target code BQAs for detection and quantification by molecular recognition, wherein the luminons bear fluorophores whose fluorescence is amplified upon binding to a BQA,
(iii) wherein primary luminons target constant region BQAs common to all proteoforms in the RTP family, independent of secondary BQAs, and
(iv) wherein biological function related BQAs are targeted by fluorophore labeled secondary luminons that code and enable quantification of multiple BQAs in an RTP, being of either positive or negative functionality,
c) detecting fluorophores in analyte:luminon complexes by either their fluorescence or fluorescence resonance energy transfer,
d) detecting specific BQAs:luminon coding assays by luminon fluorophore detection specificity,
e) computation of BQA concentration indexes based on BQA to RTP titer ratios, in which the indexes identify,
(i) the fraction of positive attribute BQAs at or above an acceptable BQA level, and/or
(ii) the fraction of negative BQAs at or below an accepted level.

7. The method of claim 6, further comprising the following steps performed in a separation sector between the BQA:luminon complex formation and the detection sector:
a) resolving of BQA:luminon complexes by either mobile affinity selection chromatography (MASC) or capillary electrophoresis (CE),
b) validating BQAs in resolved proteoforms using the retention time shift of fluorophores sequestered in BQA:luminon complexes,
c) indirect quantification of BQAs based luminon fluorophore concentration in chromatographically or electrophoretically resolved BQA:luminon complexes,
d) quantification of the RTP titer by summing the total amount of constant region primary luminon bound to the sum of all proteoforms in the RTP family as they eluted from the separation sector,
e) comparing the concentration of function targeted secondary luminon coded BQAs to constant region BQA concentration for the purpose of
(i) determining the fraction of an RTP bearing an BQA of a specific functionality, and
(ii) assessing the positive or negative therapeutic potential of a BQA by comparing that ratio to acceptable quality norms,
